(19) United States
(12) Patent Application Publication
(10) Pub. No.: US 2007/0032768 A1
(43) Pub. Date: Feb. 8, 2007

(54) Article with premium

(75) Inventors: Jason C. Cohen, Appleton, WI (US); Kenneth Thomas Borowski, Neenah, WI (US); Rodney Lee Miller JR., Shiocton, WI (US); Martin Joseph Garofalo, Menasha, WI (US); David William Koenig, Menasha, WI (US); Tatum Marie Jelenc, Chilton, WI (US); Eric Donald Johnson, Larsen, WI (US); David Roland Otts, Appleton, WI (US)

Correspondence Address: KIMBERLY-CLARK WORLDWIDE, INC., 401 NORTH LAKE STREET, NEENAH, WI, 54956, US

(73) Assignee: Kimberly- Clark Worldwide, Inc.

(21) Appl. No.: 11/198,244

(22) Filed: Aug. 5, 2005

(51) Int. Cl. A61F 13/15

(52) U.S. Cl. 604/385.01; 604/385.06

(57) ABSTRACT

An absorbent article includes an outercover, a bodyside liner, an absorbent core positioned between the outercover and the bodyside liner, and at least one premium. The premium may be associated with the inner and/or outer surface of the absorbent article. The premium may be disposed within an enclosure joined to the outercover. The premiums may be coupons, game pieces, stickers, temporary tattoos, iron on transfers, various media to convey information to the user or purchaser, or the like, or combinations thereof. The premiums and the absorbent articles may be adapted for specific users. The premiums and the absorbent articles may form part of an absorbent article system and may be located within a flexible consumer package.

### BACKGROUND OF THE INVENTION

Disposable absorbent articles, such as infant diapers, feminine hygiene products, adult incontinence products, and the like, are well known in the art. Many different product designs, features, materials, and aesthetics combinations have been suggested to meet user and consumer needs and desires. In addition, some have suggested diapers with accessories, such as, for example, powders, oils and wipes. Others have suggested diapers with advertisements.

Despite these suggestions, there is a need for disposable absorbent articles that include one or more additional items that increase the value of the article from the perspective of the user and/or the consumer.

### SUMMARY OF THE INVENTION

In response to these needs, an absorbent article has an inner surface and an outer surface, an outercover, a bodyside liner, an absorbent core positioned between the outercover and the bodyside liner, and at least one recreational or informational premium associated with the inner or outer surface.

The absorbent article may include an enclosure joined to the outercover, wherein the at least one premium is located within the enclosure. The enclosure may have a cover having at least one article image thereon. In various embodiments, the premium may include a sticker or a temporary tattoo having a premium image that is the same as the article image.

The absorbent article may also include at least one coupon, game piece, sticker, temporary tattoo, iron-on decal, toy, or the like.

In various embodiments, the outer cover may include at least one article image and the premium may include a premium image that is the same as the article image.

In various embodiments, the absorbent article may be adapted for children at a specific stage of development and the premium may also be adapted for children at the specific stage of development.

In various embodiments, the absorbent article may be adapted for a particular category of user and the premium may be adapted to include information for the particular category of user.

In another aspect, an absorbent article includes an outercover, a bodyside liner, an absorbent core positioned between the outercover and the bodyside liner, and an enclosure joined to the outercover. The enclosure includes a cover joined about a periphery to a backing to define a pocket. The pocket contains at least one premium therein.

In various embodiments, the cover may have a cover image and the premium may have a premium image that is the same as the cover image.

In various embodiments, the premium may be at least one coupon or game piece and/or at least one recreational or informational premium.

In another aspect, an absorbent article system includes a package and at least one absorbent article kit disposed within the package. Each absorbent article kit includes an absorbent article and at least one recreational or informational premium associated therewith.

In various embodiments, the package may be a consumer flexible package.

In various embodiments, the absorbent articles may include an outercover, a bodyside liner and an absorbent core positioned between the outercover and the bodyside liner, and an enclosure joined to the outercover, wherein the at least one premium is located within the enclosure.

In some embodiments, the absorbent articles may be adapted for children at specific stages of development and the premiums may be adapted for children at the specific stages of development.

In various embodiments, the absorbent article system may include a first and second absorbent article disposed within the package. The first absorbent article may include a first premium associated therewith. The second absorbent article may include a second premium associated therewith. The first premium may be different than the second premium.

In various embodiments, the package has at least one package image, the absorbent article has at least one article image, the premium has at least one premium image, and the at least one package image is the same as the at least one article image which is the same as the at least one premium image.

In various embodiments, the absorbent article system may further include a collector device for use with the various premiums.

### BRIEF DESCRIPTION OF THE DRAWINGS

FIG. 1 representatively illustrates a partially cut away, top plan view of an exemplary absorbent article in a stretched and laid flat condition with the surface of the article that contacts the skin of the wearer facing the viewer.

FIG. 2 representatively illustrates an exemplary absorbent article system.

FIG. 3 representatively illustrates exemplary absorbent article kits.

### DETAILED DESCRIPTION OF THE DRAWINGS

The present invention relates to removable premiums associated with the inner and/or outer surface of disposable absorbent articles to provide added value to said disposable absorbent articles. The added value of the premiums results from the recreational, promotional and/or informational opportunities provided to the consumer and/or user of the disposable absorbent article. By adding value, the various premiums provide additional incentive for the initial purchase and/or the repeat purchase of the disposable absorbent articles.

The premiums of the present invention may be associated with the surface of disposable absorbent articles in any suitable manner and by any suitable means. For example, the premiums may be associated with a surface of the disposable absorbent articles by being joined to the surface with adhesive. In some embodiments, the premiums may be associated with a surface by being located within enclosures which are joined to the surface. The enclosures may be formed by covers joined to backings to define pockets.

The disposable absorbent articles of the present invention will be described in terms of a diaper adapted to be worn by infants about the lower torso. It is understood that the disposable absorbent articles of the present invention are equally applicable to other disposable absorbent articles such as adult incontinent products, training pants, feminine care products, and the like.

As used herein, the term “join”, and derivatives thereof, encompasses configurations wherein an element is directly secured to another element by affixing the element directly to the other element and configurations wherein the element is indirectly secured to the other element by affixing the element to intermediate member(s) which in turn are affixed to the other element.

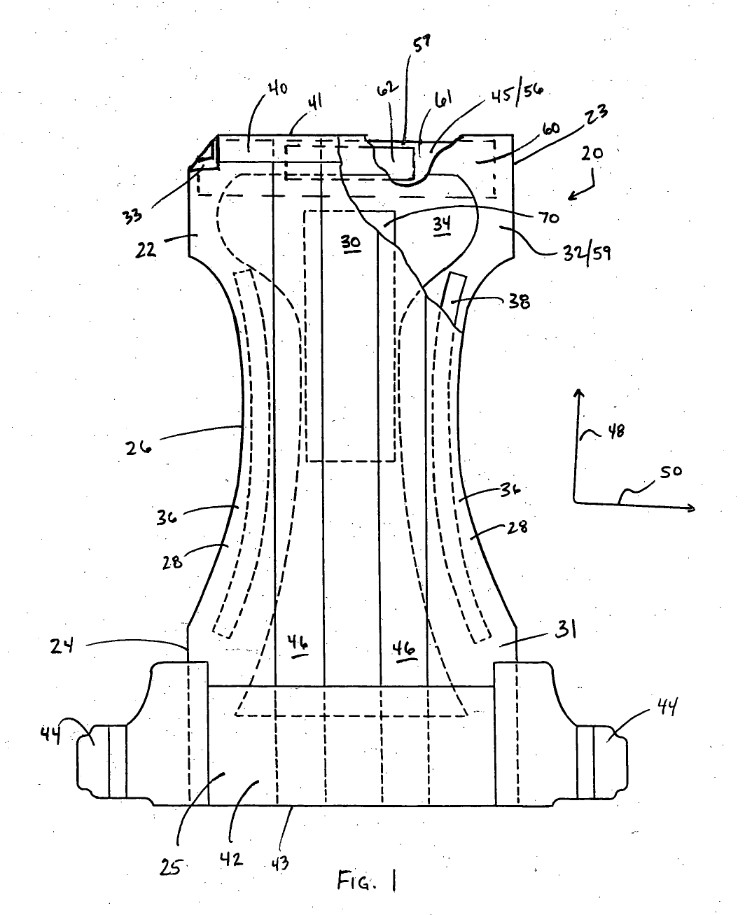

FIG. 1 representatively illustrates an embodiment of an absorbent article 20 of the present invention. The surface of the article which contacts the wearer is facing the viewer. The absorbent article 20 defines a front portion 22, a rear portion 24 and a crotch portion 26 connecting the front portion 22 and the rear portion 24. The front portion 22 defines a front waist region 23 and includes a front waist edge 41. The rear portion 24 defines a rear waist region 25 and includes a rear waist edge 43. The absorbent article 20 also defines a longitudinal direction 48 and a lateral direction 50. The absorbent article 20 includes a bodyside liner 30, an outer cover 32 and an absorbent core 34 located between the bodyside liner 30 and the outer cover 32. The absorbent article 20 includes an inner surface 31 generally oriented towards the skin of the wearer and an outer surface 33 generally oriented towards the clothing of the wearer. The absorbent article 20 may also include one or more premiums 62 associated with the bodyside liner 30 and/or the outer cover 32 and/or the inner surface 31 and/or the outer surface 33.

The absorbent article 20 may also include one or more enclosures 60. The enclosures 60 may include at least one cover 56 joined about a cover periphery 57 to at least one backing 59. The enclosures 60 define one or more pockets 61 wherein each pocket 61 may have one or more premiums 62 disposed therein.

As used herein, the term “premium” refers to objects that are provided in addition to disposable absorbent articles, are adapted to add value to the disposable absorbent articles, are associated with individual disposable absorbent articles and are adapted to separate from the disposable absorbent article but are not adapted to be used as part of the hygienic process generally associated with the disposable absorbent articles. For example, wipes, lotions, powders, disposal bags, medicaments, and the like are not considered premiums as used herein.

As used herein, reference to a front portion refers to that part of the absorbent article which is generally located on the front of a wearer when in use. Reference to a front waist region refers to that part of the front portion which is located generally near the waist opening. Reference to the rear portion refers to the portion of the article generally located at the rear of the wearer when in use. Reference to a rear waist region refers to that part of the rear portion which is located generally near the waist opening. Reference to the crotch portion refers to that portion which is generally located between the legs of the wearer when in use. In some embodiments, the liner forms the inner surface. In some embodiments, the outer cover forms the outer surface.

The crotch portion 26 has opposite longitudinal side portions 28 which may include a pair of elasticized, longitudinally-extending leg cuffs 36. The leg cuffs 36 are generally adapted to fit about the legs of a wearer in use and serve as a mechanical barrier to the lateral flow of body exudates. The leg cuffs 36 may be elasticized by a pair of leg elastics 38. The absorbent article 20 may further include a front waist elastic 40 and/or a rear waist elastic 42. The rear portion 24 of the absorbent article 20 may further include a fastening means 44 which is intended to hold the absorbent article 20 about the waist of the wearer when in use. The absorbent article 20 may also include a pair of containment flaps 46 which extend longitudinally along the absorbent article 20 and are also adapted to provide a barrier to the flow of body exudates. It should be recognized that individual components of the absorbent article 20, such as the elastic members, may be optional depending upon the intended use of the absorbent article 20.

The bodyside liner 30 of the absorbent article 20 suitably presents a bodyfacing surface which is intended to be worn adjacent the body of the wearer and is compliant, soft feeling and nonirritating to the wearer's skin. A suitable bodyside liner 30 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

The outer cover 32 of the absorbent article 20 may suitably be composed of a material which is either liquid permeable or liquid impermeable. It is generally preferred that the outer cover 32 be formed from a material which is substantially impermeable to fluids. For example, a typical outer cover can be manufactured from a thin plastic film or other flexible liquid-impermeable material. For example, the outer cover 32 may be formed from a polyethylene film. If it is desired to present the outer cover 32 with a more clothlike feeling, the outer cover 32 may comprise a polyethylene film having a nonwoven web laminated to the outer surface thereof, such as a spunbond web of polyolefin fibers. Methods of forming such clothlike outer covers are known to those skilled in the art.

The bodyside liner 30 and outer cover 32 are generally joined together so as to form a pocket in which the composite absorbent core 34 is located. The bodyside liner 30 and outer cover 32 may be joined directly to each other around the outer periphery of the absorbent article 20 by any means known to those skilled in the art such as, for example, adhesive bonds, sonic bonds or thermal bonds. Such bonding means may also be suitable for attaching other components of the composite absorbent core and absorbent article of the present invention together. The leg cuffs 36 are suitably formed by portions of the outer cover 32, and/or bodyside liner 30, which extend beyond the longitudinal sides of the composite absorbent core 34. Naturally, the leg cuffs 36 may also be formed from separate materials which are attached to the outer cover 32 and/or bodyside liner 30.

The leg cuffs 36 may include leg elastics 38. Waist elastics 40 and 42 may also be provided. The leg elastics 38 are arranged to draw and hold the absorbent article 20 against the legs of the wearer. The waist elastics 40 and 42 are also arranged to draw and hold the absorbent article 20 against the wearer. Materials suitable for use in forming leg elastics 38 and waist elastics 40 and 42 are known to those skilled in the art. Exemplary of such materials are strands or ribbons of a polymeric, elastomeric material which are adhered to the absorbent article 20 in a stretched position, or which are attached to the absorbent article while the article is pleated, such that elastic constrictive forces are imparted to the absorbent article 20. In a particular aspect of the invention, the elastics may be composed of individual strands of LYCRA which are available from INVISTA Co., a business having offices in Wichita, Kans., USA.

The leg elastics 38 and waist elastics 40 and 42 may have any configuration which provides the desired performance. For example, the leg elastics 38 and waist elastics 40 and 42 may comprise a single strand of elastic material, or may comprise several parallel or non-parallel strands of elastic material. The leg elastics 38 may be generally straight or optionally curved to more closely fit the contours of the legs and buttocks of the wearer and better contain bodily exudates. The leg elastics 38 and waist elastics 40 and 42 may be attached to the absorbent article 20 in any of several ways which are well known to those skilled in the art. For example, the elastics may be ultrasonically bonded, thermally bonded or adhesively bonded to the absorbent article 20.

The fastening means 44 are typically applied to the corners of the rear portion 24 of the absorbent article 20 to provide a means for holding the article 20 on the wearer. Suitable fastening means 44 are well known to those skilled in the art and can include tape tab fasteners, hook and loop fasteners, mushroom and loop fasteners, snaps, pins, belts, and the like, and combinations thereof. Typically, the fastening means 44 are configured to be refastenable. It should also be understood that it may be possible to dispense with the fastening means 44 in an absorbent article having a given design configuration.

In some embodiments, the fastening means 44 may be adapted to engage or otherwise join with a fastener landing material 45. Typically, the fastener landing material 45 is a loop material joined to the outercover 32 in the front waist region 23 and is adapted to engage hook-type fastening means 44. In alternative embodiments, the fastener landing material 45 may be a film adapted to engage with tape tab fastening means 44.

The absorbent core 34 is positioned between the bodyside liner 30 and the outer cover 32 to form the absorbent article 20. The absorbent core 34 is generally conformable and capable of absorbing and retaining body exudates. It should be understood that, for the purpose of the present invention, the absorbent core 34 may comprise a single, integral piece of material or, alternatively, may comprise a plurality of individual separate pieces of material which are operably assembled together.

The absorbent core 34 may have any of a number of shapes and sizes. The absorbent core 34 may suitably comprise various types of wettable, hydrophilic fibrous materials. Examples of suitable materials include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester and polyamide fibers; and synthetic fibers composed of a nonwettable thermoplastic polymer, such as polypropylene fibers, which have been hydrophilized by appropriate means known to those skilled in the art. The absorbent core 34 may also comprise selected blends of the various types of fibers mentioned above. The absorbent core 34 may include a matrix of hydrophilic fibers, such as a web of cellulosic fibers, mixed with particles of a high-absorbency material such as that commonly known as superabsorbent material.

The absorbent article 20 of the present invention may also contain a surge portion 70 to advantageously improve the overall fluid intake rate of the absorbent core 34. The surge portion 70 is typically less hydrophilic than the absorbent core 34 and is configured to collect and temporarily hold fluid surges. This configuration can also help prevent fluid exudates from pooling and collecting on portions of the absorbent core 34.

In various embodiments, one or more enclosures 60 may be associated with, for example, the outercover 32, the bodyside liner 30, the inner surface 31, the outer surface 33, or combinations thereof. The enclosures 60 may be comprised of at least one cover material 56 and at least one backing material 59. The cover materials 56 have a cover periphery 57. One or more of the cover materials 56 may be joined with one or more backing materials 59 about the cover periphery 57 to form the enclosure 60. The enclosure 60 defines a pocket 61 which may include one or more premiums 62 disposed therein. The premiums 62 may be located between one or more of the cover materials 56 and one or more of the backing materials 59.

In various embodiments, the enclosures 60 may be associated with the bodyside liner 30 and/or the outercover 32 and/or the inner surface 31 and/or the outer surface 33 of the absorbent article 20 by any suitable means. Desirably, the backing material 59 may be joined to the absorbent article 20 thereby leaving the cover material 56 accessible to the caregiver or user.

In various embodiments, one or more components of the enclosures 60 may be multifunctional. As used herein, the term “multifunctional” describes one or more components of an enclosure 60 that are also components of the absorbent article. 20. In other words, the one or more components are functional in the article for reasons other than forming portions of the enclosures 60. For example, an enclosure 60 comprising the fastener landing material 45 functioning as the cover material 56 and/or the outercover 32 functioning as the backing material 59 would be considered multifunctional.

For example, the waist elastics 40 and/or 42 may serve as the cover material 56 and the bodyside liner 30 may serve as the backing 59. In these embodiments, the waist elastics 40 and/or 42 are generally joined about the periphery 57 to the bodyside liner 30 in the front waist region 23 and/or rear waist region 25 so as to form the enclosure 60. The enclosure 60 defines a pocket 61 which may contain one or more premiums 62 disposed therein.

FIG. 1 representatively illustrates an enclosure 60 being formed by the fastener landing material 45 (functioning as the cover material 56) and the outercover 32 (functioning as the backing material 59). In this embodiment, the fastener landing material 45 is joined about the periphery 57 to the outercover 32 in the front waist region 23 to form the enclosure 60. The enclosure 60 defines a pocket 61 which contains one or more premiums 62 therein.

In various embodiments, the enclosures 60 may contain additional layers as necessary to achieve the desired functional and/or aesthetic objectives of the designer. For example, additional layers may be added to create a cloth-like feel and/or to add elasticity and/or to alter vapor permeability and/or to alter liquid impermeability, and the like.

The materials used to make the covers 56 and/or backings 59 may be any suitable material described herein. For example, the covers 56 and/or backings 59 may be manufactured from a wide selection of web materials, such as porous foams, reticulated foams, plastic films, natural fibers, synthetic fibers, or a combination of natural and synthetic fibers. Various woven and nonwoven fabrics may be used for the covers 56 and/or the backings 59. For example, the covers 56 and/or the backings 59 may be composed of a meltblown or spunbonded web of polyolefin fibers and/or bonded carded web of natural and/or synthetic fibers. The covers 56 and/or the backings 59 may suitably be composed of a material which is either liquid permeable or liquid impermeable. The covers 56 and/or the backings 59 may suitably be composed of a material which is either vapor permeable or liquid permeable. The covers 56 and/or the backings 59 may be formed from a polyethylene film having a nonwoven web laminated to the outer surface thereof, such as a spunbonded web of polyolefin fibers. The covers 56 and/or the backings 59 may be extensible and may be elastic.

The enclosures 60 may have one or more seams. The seams may include pull open seams such as, for example, zipper seams, lap seams, lines of weakness seams, and the like, and combinations thereof. The seams may include any closure means that allows a user to manually transition the enclosures 60 from a closed condition to an open condition. Once in the open condition, the one or more premiums 62 contained within the enclosures 60 are accessible to the user. The one or more seams are desirably located in the cover material 56 but may alternatively or additionally be located in the backing material 59.

As used herein, the term “pull open seam” refers to seams that can be transitioned from a closed condition to an open condition by manually applying force to the seam. Often this force involves pulling the material(s) on opposite sides of a seam in opposing directions to effect a separation.

As used herein, the term “zipper seam” refers to self-mating rib and flange seams such as are commonly used with sandwich bags. A suitable zipper seam is described in U.S. Pat. No. 6,544,604 issued Apr. 15, 2003 to Galkiewicz et al.

As used herein, the term “lap seam” refers to the joining of two or more materials in face to face relation, i.e., wherein one material “overlaps” another material. The overlapping material may or may not be joined by any suitable means, such as, for example, adhesive.

As used herein, the term “line of weakness” seam refers to a seam created in one or more pieces of material by partial pressure cutting, partial ultrasonic cutting, partial thermal deformation, mechanical thinning, perforating or other processes as are known in the art. The line of weakness seam may extend, at least partially, through the given material(s).

As used herein, the term “perforating” means to make one or more holes, slits, apertures, voids, or the like, or combinations thereof through one or more materials to facilitate separation of the one or more materials.

As used herein the term “closed condition” describes the enclosures 60 when the one or more premiums located therein are not accessible to a user without destroying the enclosure 60 and/or destroying the absorbent article 20 to which the enclosure 60 is joined and/or separating one or more seams.

As used herein the term “open condition” describes the enclosure 60 after the one or more seams are separated and when the one or more premiums 62 located therein are accessible to a user.

In various embodiments, the seams may be adapted to be resealable such that a user may transition the enclosure 60 from the closed condition to the open condition by separating the seam and then transition the enclosure 60 from the open condition to the closed condition by re-joining the seam. In other embodiments, the seams may be adapted such that once the seam is separated, the seam may not be re-joined and the enclosure 60 cannot be resealed.

In various embodiments, the seams may be adapted such that once the seam is separated, the enclosure 60 remains multifunctional as an absorbent article component. For example, the enclosure 60 may also be functional as a waist elastic having elasticity in the lateral direction 50. The enclosure 60 may include a seam generally aligned in the lateral direction 50. When the user separates the seam, the waist elastic maintains its functional elasticity. In yet other embodiments, the seams may be adapted such that once the seam is separated, the enclosure 60 is no longer multifunctional.

In various embodiments, the one or more seams may be made more visible or prominent through the use of one or more seam indicators. The seam indicators may include printing, embossing, bonding, or the like, or combinations thereof of one or more indicia proximate the seams. The seam indicators are adapted to draw the caregiver's or user's attention to the seams and may include shapes, symbols, text, graphics, or the like, or combinations thereof.

In various embodiments, the one or more seams may include a tamper evident device, such as, for example, a color-change element or a text alignment element as are known in the art.

In various embodiments, the premiums 62 may be self contained within a packet. As used herein, the term “self contained” refers to premiums that are encased in a packet in addition to the cover material and the backing material.

The packet may include one or more sub-covers and/or one or more sub-backings. The sub-covers may be joined about the periphery to the sub-backings to define a second pocket. The premium 62 may then be contained within the second pocket within the packet. The packet may then be contained within the pocket 61 within the enclosure 60. In various embodiments, a self contained premium may be desirable for reasons of sterility, moisture retention, safety, light avoidance, air avoidance, thermal stability, and the like.

In various embodiments, the premium 62 may be free to move within the enclosure 60. This may be advantageous in that elastic and/or extensible materials may be used to create the cover material 56 and/or the backing material 59 thereby creating an elastic and/or extensible enclosure 60. As the absorbent article 20 is used, the outercover 32 and/or the bodyside liner 30 and/or the front waist elastic 40 may extend and/or contract without inhibition from the premium 62 contained within the enclosure 60 or without inhibition from the materials used to create the enclosure 60.

Various suitable enclosures 60 are described in commonly assigned U.S. application Ser. No. ______ to Cohen et al. (atty. Docket 21,602) filed Aug. 5, 2005 and entitled “Absorbent Articles With Enclosures”, the entirety of which is incorporated herein by reference where not contradictory.

In various embodiments, a kit comprises a disposable absorbent article and at least one premium associated therewith. In various embodiments of the kits, the premiums may be presented within an enclosure on or near a surface of the disposable absorbent article. In other embodiments, the premiums may be presented directly on one or more surfaces of the disposable absorbent article.

In various embodiments, the disposable absorbent articles may be adapted for use by a specific category of user, such as, for example, newborn children, infants, toddlers, females, males, adults, menstruating women, and the like, as is known in the art. A specific category may be defined by multiple descriptors such as, for example, male infants. Adaptation for a specific category of user is known in the art. For example, a disposable absorbent article may be adapted for a specific category by sizing the product based on the estimated size of the intended user. Adaptation may include shaping the product to conform with male or female genitalia.

The absorbent capacity and Theological demands of the disposable absorbent article may also be adapted for a specific category of user as is known in the art. For example, a feminine hygiene article requires different absorption characteristics and capacity than does an infant diaper or adult incontinence article. The aesthetics of a disposable absorbent article may also be adapted for a specific user and/or purchaser. For example, the graphics for an infant diaper may be adapted to include popular cartoon characters. Similarly, aesthetics may be adapted on training pants and/or adult incontinence products to mimic the appearance of cloth underwear.

A disposable diaper, for example, may be adapted for use by newborn children as is known in the art, for example, by sizing the diaper to fit newborn children, using softer materials, providing umbilical features, providing graphics that are pleasing to a newborn child and a caregiver, and the like, and combinations thereof.

In various embodiments, the premiums associated with the disposable absorbent articles may be adapted to appeal to the specific category of user and/or purchaser to which the absorbent article is adapted.

In categories wherein the purchaser of the disposable absorbent articles is different than the user of the disposable absorbent article, a disposable absorbent article may be adapted to a specific user and a premium associated with the disposable absorbent article may be adapted to appeal to the consumer of the disposable absorbent article and/or the caregiver using the disposable absorbent article. For example, a disposable diaper may be adapted for use by a newborn child and the associated premium may be adapted to appeal to the purchaser of newborn diapers.

In various embodiments, the premiums may be promotional premiums. As used herein, the term “promotional premium” refers to objects that are provided in addition to disposable absorbent articles, are adapted to further the sale of the disposable absorbent article and/or other merchandise through advertising, publicity, or discounting, are associated with individual disposable absorbent articles, and are adapted to separate from the disposable absorbent article. Examples of promotional premiums include, for example, coupons, game pieces, prizes, advertisements, and the like, and combinations thereof.

As used herein, the term “coupon” refers to a certificate or note entitling the bearer thereof to exchange the certificate or note for select goods or exchange the certificate or note for the option to purchase select goods at a discounted price. As used herein, the term “game piece” refers to a token, marker or the like that is used in conjunction with a promotion. In various embodiments, one or more game pieces may be redeemed for cash or prizes or both. As used herein, the term “prizes” refers to cash, checks, concert tickets, and the like, and combinations thereof.

In various embodiments, the disposable absorbent article may be an infant diaper and the promotional premium may be a coupon that is redeemable, at least in part, for additional infant diapers. The addition of the coupon provides incentive for the initial purchase of the infant diapers and/or the repeat purchase of the infant diapers to which the coupon is directed. In other embodiments, the coupon may be redeemable, at least in part, for training pants, toiletries, toys, clothing, and the like, and combinations thereof. In these embodiments, the addition of the coupon provides incentive for the purchase of another product, such as, for example, those listed above.

In various embodiments, the promotional premiums associated with the disposable absorbent articles may be adapted to appeal to the specific category of user and/or purchaser to which the absorbent article is adapted thereby creating a promotional kit. The promotional premiums may be adapted, for example, by providing coupons redeemable, at least in part, for items likely to be of interest to a given category of user and/or purchaser.

For example, a feminine hygiene article may be adapted for use by menstruating females as is known in the art. A promotional premium, adapted to appeal to menstruating females, may be associated with the feminine care article. For example, the promotional premium may be a coupon for other feminine care articles such as, for example, tampons, pads, and the like. Alternatively or additionally, the promotional item may include a coupon for makeup, hair care products, skin care products, toiletries, salon services, and the like, and combinations thereof.

In another example, a disposable infant diaper may be adapted for use by infants as is known in the art. A promotional premium, adapted to appeal to purchasers of infant diapers, may be associated with the diaper. For example, the premium may be a coupon for infant care products such as, for example, diapers, baby wipes, toiletries, changing pads, and the like, and combinations thereof. In various embodiments, the premium may be a coupon for baby food, clothing, toys, books, and the like, and combinations thereof.

In another example, a disposable absorbent article may be adapted for use by incontinent adults as is known in the art. A promotional premium may be associated with the adult incontinence product that is adapted to appeal to incontinent adults. For example, the promotional premium may be one or more game pieces associated therewith. The game pieces may be adapted such that a single game piece entitles the bearer to redeem the game piece for cash and/or prizes. Alternatively or additionally, the game pieces may be adapted such that multiple game pieces are required to entitle the bearer of the game pieces to redeem the game pieces for cash and/or prizes.

In various embodiments, the premiums may be recreational premiums. As used herein, the term “recreational premium” refers to objects that are provided in addition to disposable absorbent articles, are adapted to entertain a user and/or a purchaser of the disposable absorbent articles, are associated with individual disposable absorbent articles, and are adapted to separate from the disposable absorbent article. Examples of recreational premiums include, for example, toys, stickers, temporary tattoos, trading cards, comic books, iron-on decals, and the like, and combinations thereof.

In various embodiments, the recreational premiums provided with the disposable absorbent articles may be adapted to appeal to the specific category of user and/or purchaser to which the absorbent article is adapted, thereby creating a recreational kit.

For example, a disposable infant diaper or training pant may be adapted for use by infants or toddlers as is known in the art. A recreational premium may be associated with the diaper or training pant that is adapted to appeal to purchasers and/or users of diapers or training pants. For example, the recreational premium may be one or more stickers, tattoos, iron on transfers, and the like, and combinations thereof. In some embodiments, the recreational premiums may depict images of cartoon characters. In some embodiments, the disposable infant diaper or training pant may be adapted for use by infants or toddlers by including images of the same cartoon characters as depicted by the recreational premiums.

In another example, a disposable swim pant may be adapted for use by infants and toddlers as is known in the art. A recreational premium may be associated with the swim pant wherein the recreational premium is adapted to appeal to infants and toddlers. For example, the recreational premium may be a toy suitable for use in and around the water, such as, for example, a compressed foam character provided in a water soluble capsule. The recreational kit provides the swim pant and the toy, both adapted to infants and toddlers, thereby increasing the value of the swim pant to the user and/or caregiver.

In various embodiments, a diaper or training pant may be adapted, for infants or toddlers by providing images of one or more cartoon characters on the article, the package, or both. Likewise, the recreational premium may include images of the same or related cartoon characters. For example, an image of a popular cartoon character may be depicted, via printing or other known means, on an absorbent article and/or a package containing absorbent articles. The diaper or training pant may further include one or more recreational premiums depicting an image of the same cartoon character. The depiction can take any suitable form such as, for example, stickers, pictures, toys, and the like. The addition of the premium to the diaper or training pant adds entertainment value to the user and/or purchaser. The coordination of the premium with the images on the diaper or training pant creates an entertaining game for the infant or toddler.

In various embodiments, the premiums may be informational premiums. As used herein, the term “informational premium” refers to objects that are provided in addition to disposable absorbent articles, are adapted to communicate information to the user and/or consumer of the absorbent article, are associated with individual disposable absorbent articles, and are adapted to separate from the disposable absorbent article. Examples of informational premiums include cards, paper, electronic media, or other suitable media capable of storing and conveying information. The informational premiums do not include information provided primarily as part of a promotion.

In various embodiments, the informational premiums associated with the disposable absorbent articles may be adapted to appeal to the specific category of user and/or purchaser to which the absorbent article is adapted thereby creating an informational kit. The informational premiums may be adapted, for example, by providing information likely to be of interest to a given category of user and/or purchaser.

For example, a feminine hygiene article may be adapted for use by menstruating females as is known in the art. An informational premium may be associated with the feminine care article that is adapted to interest menstruating females. For example, the informational premium may be a card containing information about menstruation, women's health, motivational or inspirational statements, jokes, and the like. The informational premium may additionally or alternatively include addresses for web sites available on the Internet. The web sites may contain information related to issues of interest for users of feminine hygiene products.

In another example, a disposable infant diaper or training pant may be adapted for use by infants and/or children as is known in the art. The diaper or pant may be adapted for an infant or child at a specific age, size and/or stage of development. An informational premium may be associated with the diaper that is adapted to interest purchasers of infant diapers and/or training pants of that specific age, size and/or stage of development. For example, the informational premium may be a card containing information about children's health, such as sleep habits, thumb sucking, teething, skin health, toilet training, and the like, and combinations thereof. In another example, a diaper may be adapted for use by newborn children by including an umbilical feature. The informational premium may be adapted to purchasers of newborn diapers. For example, the information provided with the informational premium may relate to issues such as, for example, umbilical care, sleeping positions and nursing.

The informational premium may additionally or alternatively contain information describing activities that are suitable for caregivers and users of diapers and training pants. The activities may be adapted for a child at a specific age, size and/or stage of development. For example, the activities may be adapted to promote interaction between the child and the caregiver. The information premium may additionally or alternatively include addresses for web sites available on the Internet. The web sites may contain information related to issues of interest for purchasers of diapers and/or training pants.

In another example, a disposable absorbent article may be adapted for use by incontinent adults as is known in the art. An informational premium may be associated with the adult incontinence product that is adapted to appeal to incontinent adults. For example, the informational premium may be a card containing information about incontinence, aging, skin health, and the like. The information premium may additionally or alternatively include addresses for web sites available on the Internet. The web sites may contain information related to issues of interest for users of adult incontinence products.

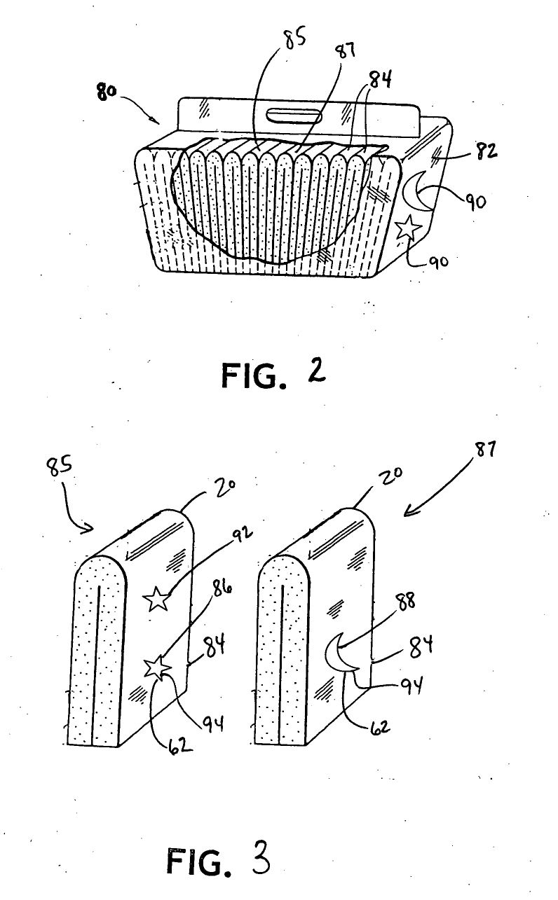

Referring now to FIGS. 2 and 3, a disposable absorbent article system 80 may be provided comprising a package 82 and one or more of the absorbent article kits 84 disposed therein. In various embodiments, the package 82 may comprise a rigid material such as cardboard, molded plastic, and the like. In various embodiments, the package 82 may be a flexible consumer package. As used herein, the term “flexible consumer package” refers to non-rigid containers, such as polyethylene bags, that are adapted to contain disposable absorbent articles and are adapted to be presented to a consumer. The one or more absorbent article kits 84 include at least one premium 62 associated with a surface of a disposable absorbent article 20 to add value to the article. Alternatively or additionally, the package 82 may include one or more disposable absorbent articles 20 that have no premium 62 associated therewith.

In some embodiments, the package 82 may include at least a first disposable absorbent article kit 85 and a second disposable absorbent article kit 87 wherein the first and second disposable absorbent article kits 85 and 87 include premiums 62 associated therewith. The first disposable absorbent article kit 85 may include a first premium 86 associated therewith. The second disposable absorbent article kit 87 may include a second premium 88 associated therewith.

The first and second premiums 86 and 88 may be the same type of premium. For example, the first premium 86 may comprise a sticker and the second premium 88 may comprise a sticker. The images depicted on the first premium 86 and the second premium 88 may or may not be the same. For example, the first premium 86 may be a sticker depicting the image of a frog and the second premium 86 may be a sticker depicting the image of a ladybug. In another example, the first and second premiums 86 and 88 may both be stickers depicting the same image.

The first and second premiums 86 and 88 may be different types of premiums. Different types of premiums include, for example, coupons, game pieces, toys, stickers, temporary tattoos, trading cards, comic books, iron-on decals, cards containing information, and the like. For example, the first premium 86 may comprise a coupon and the second premium 88 may comprise a toy. In another example, the first premium 86 may comprise a toy and the second premium 88 may comprise sticker. The images depicted on the first premium 86 and the second premium 88 may or may not be the same. For example, the first premium 86 may be a sticker depicting a duck and the second premium 88 may be a toy depicting the image of a crab or the toy may be in the shape of a crab. In another example, the first premium 86 may be a sticker depicting the image of a frog and the second premium 86 may be a toy depicting the image of a frog or the toy may be in the shape of a frog.

As used herein, the term “same as” refers to images that depict a common character, shape, and the like. One image may be a different size and may have subtle differences and still be considered the same as a second image. For example, a large star image would be the same as a small star image because both depict a star. In another example, the image of a cartoon character clapping would be the same as an image of the cartoon character running because both depict the same cartoon character.

Any of the premiums described herein may be combined with any of the others to produce various disposable absorbent article systems. For example, a package of disposable diapers may include premiums associated therewith. One or more of the diapers may include a coupon and/or one or more of the diapers may include a toy and/or one or more of the diapers may include a game piece and/or one or more of the diapers may include a sticker.

In various embodiments, the disposable absorbent article system 80 may include a package 82 having one or more package images 90 visible thereon. For example, the package 82 in FIG. 2 has two package images 90, one of a moon and one of a sun.

In various embodiments, the disposable absorbent article system 80 may include at least one disposable absorbent article kit 84 comprising a disposable absorbent article 20 having one or more article images 92 visible thereon. For example, the disposable absorbent article kit 85 in FIG. 3 has an article image 92, a star.

In various embodiments, the disposable article system 80 may include at least one disposable article kit 84. The disposable article kit 84 includes disposable article 20 and at least one premium 62 associated therewith. The at least one premium 62 may comprise one or more premium images 94. For example, the first absorbent article kit 85 of FIG. 3 includes a first premium 86 having a premium image 94. The premium 86 is a separate removable sticker and the premium image 94 is that of a star. In various embodiments, the premium image 94 may also be depicted by a toy shaped like a star or a temporary tattoo comprising the image of a star, or the like.

In various embodiments, the premium image 94 may be the same as one or more of the article images 92. This is illustrated in absorbent article kit 85 in FIG. 3. The premium image 94 is a star and the article image 92 is a star.

In various embodiments, the premium image 94 may be the same as one or more of the package images 90. This is illustrated in absorbent article kits. 85 and 87 of FIG. 3. The absorbent article kit 85 has a premium image 94 of a star and one of the package images 90 is a star, as illustrated in FIG. 2. Absorbent article kit 87 has a premium image 94 of a moon and one of the package images 90, as illustrated in FIG. 2, is a moon.

In various embodiments, the premium image 94 may be the same as one or more of the article images 92 which may be the same as one or more of the package images 90.

Suitable images 90 and/or 92 and/or 94 may include morphics as described in commonly assigned U.S. application Ser. No. ______ to Grimer et al. (atty. Docket 21,850) filed Jul. 28, 2005 and entitled “Hygiene System”, the entirety of which is incorporated herein by reference where not contradictory.

The disposable article system 80 may also include one or more collector devices such as, for example, posters, charts, maps, books, containers, and the like, and combinations thereof. The collector devices may be used to keep and/or organize the premiums acquired over time.

For example, a collector device may be a poster adapted to receive premiums in the form of stickers. The stickers may include various characters and a user may collect all the available characters and position the characters on the poster. In another example, the collector device may be game board suitable for collecting premiums in the form of game pieces. In another example, the collector device may be a progress chart useful in potty training. A premium in the form of a sticker may be placed on the chart indicating successful use of the toilet.

While the invention has been described in detail with respect to specific embodiments thereof, it will be appreciated that those skilled in the art, upon attaining an understanding of the foregoing will readily appreciate alterations to, variations of and equivalents to these embodiments. Accordingly, the scope of the present invention should be assessed as that of the appended claims and any equivalents thereto.

1. An absorbent article having an inner surface and an outer surface, the absorbent article comprising,
an outercover,
a bodyside liner,
an absorbent core positioned between the outercover and the bodyside liner, and
at least one recreational or informational premium associated with the inner or outer surface.

2. The absorbent article of claim 1 further comprising an enclosure joined to the outercover, wherein the at least one premium is located within the enclosure.

3. The absorbent article of claim 2 wherein the enclosure comprises a cover having at least one article image thereon and wherein the at least one premium comprises a sticker or a temporary tattoo having a premium image, the premium image being the same as the article image.

4. The absorbent article of claim 1 further comprising at least one coupon or game piece.

5. The absorbent article of claim 1 wherein the premium comprises at least one sticker or temporary tattoo.

6. The absorbent article of claim 5 wherein the outer cover includes at least one article image, the premium has a premium image and the article image is the same as the premium image.

7. The absorbent article of claim 1 wherein the absorbent article is adapted for children at a specific stage of development and wherein the premium is adapted for children at the specific stage of development.

8. The absorbent article of claim 1 wherein the absorbent article is adapted for a particular category of user and wherein the premium comprises information adapted for the particular category of user.

9. An absorbent article comprising,
an outercover,
a bodyside liner,
an absorbent core positioned between the outercover and the bodyside liner, and
an enclosure joined to the outercover, the enclosure comprises a cover joined about a periphery to a backing to define a pocket, the pocket containing at least one premium therein.

10. The absorbent article of claim 9 wherein the cover has a cover image and the premium has a premium image, wherein the cover image is the same as the premium image.

11. The absorbent article of claim 9 wherein the premium is at least one coupon or game piece.

12. The absorbent article of claim 9 wherein the premium is at least one recreational or informational premium.

13. An absorbent article system comprising,
a package, and
at least one absorbent article kit disposed within the package, each absorbent article kit comprising an absorbent article having at least one recreational or informational premium associated therewith.

14. The absorbent article system of claim 13 wherein the package is a flexible consumer package.

15. The absorbent article system of claim 13 wherein the absorbent articles comprise,
an outercover,
a bodyside liner,
an absorbent core positioned between the outercover and the bodyside liner, and
an enclosure joined to the outercover, wherein the at least one premium is located within the enclosure.

16. The absorbent article system of claim 13 wherein the absorbent articles are adapted for children at a specific stage of development and wherein the premiums are adapted for children at the specific stage of development.

17. The absorbent article system of claim 13 comprising,
a first absorbent article disposed within the package, the first absorbent article comprising a first premium associated therewith,
a second absorbent article disposed within the package, the second absorbent article comprising a second premium associated therewith, the first premium being a different type than the second premium.

18. The absorbent article of claim 17, wherein the first premium comprises a toy and the second premium comprises a sticker.

19. The absorbent article system of claim 13 wherein the package has at least one package image, the absorbent article has at least one article image, the premium has at least one premium image, and the at least one package image is the same as the at least one article image which is the same as the at least one premium image.

20. The absorbent article system of claim 13 further comprising a collector device.
